(19) United States
(12) Patent Application Publication
(10) Pub. No.: US 2002/0081271 A1
(43) Pub. Date: Jun. 27, 2002

(54) Composition containing a retinoid and a benzotriazole silicone

(75) Inventors: Guenaelle Martin, Paris (FR); Philippe Touzan, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/984,492

(22) Filed: Oct. 30, 2001

(30) Foreign Application Priority Data
Oct. 30, 2000 (FR) 00 13938

(51) Int. Cl. A61K 7/42; A61K 31/695

(52) U.S. Cl. 424/59; 514/63

(57) ABSTRACT

The present invention relates to a composition containing a retinoid, in particular retinol, and a specific benzotriazole silicone.

### BACKGROUND OF THE INVENTION

1. Field of the Invention

The present invention relates to compositions, preferably cosmetic and/or dermatological compositions, more preferably compositions intended for caring for the skin, comprising, preferably in a physiologically acceptable medium, a retinoid and a benzotriazole silicone. The uses of such compositions similarly makes up a part of the invention, as does their preparation.

2. Discussion of the Background

Retinoid-based cosmetic and/or dermatological compositions have experienced significant growth in recent years, in particular for the treatment of acne and cutaneous imperfections, because of the ability of retinoids to regulate the differentiation of keratinocytes, and for the prevention and treatment of certain signs of intrinsic or photoinduced cutaneous ageing, such as the formation of wrinkles and the loss of firmness of the skin, due in particular to their ability to modulate the synthesis of collagen.

Among the derivatives of the retinoids family, retinol, also known under the name of vitamin A, is of very particular advantage. This is because retinol is a natural endogenous constituent of the human body which is well tolerated on application to the skin up to higher levels than retinoic acid.

However, when it is introduced into a cosmetic or dermatological composition intended for topical application, the retinol quickly decomposes under the effect of light, oxygen, metal ions, oxidizing agents or water and in particular under the effect of a rise in temperature. The thermal decomposition of retinol has formed the subject of a study published in J. Soc. Cosm. Chem., 46, 191-198 (July-August 1995).

To overcome these problems, provision has in particular been made to use retinyl esters, which are relatively more stable than retinol. However, the stability of these compounds in cosmetic compositions is still unsatisfactory, in particular as regards esters of retinol with an acid comprising a fatty chain.

The inventors have noticed in particular that the introduction of some UVA screening agents into cosmetic compositions comprising retinol or one of its esters leads to decomposition of the retinoid over time, without the reasons for this decomposition being able to be clearly identified.

This is particularly unfortunate as regards compositions intended for the prevention or treatment of cutaneous signs of photoageing, in so far as it is often advantageous to combine, in these compositions, the biological effects of the retinoid, in particular with regard to the synthesis of collagen, with the effects of the sunscreens which absorb UVA radiation. This is because these screening agents make it possible to help the cells defend themselves against the excess of photoinduced free radicals and to prevent the decomposition of the collagen fibers due to UVA radiation. They therefore have complementary antiaging effects to those of the retinoid.

### OBJECT OF THE INVENTION

The importance in having available compositions, in particular cosmetic compositions, comprising both a retinoid and a UV screening agent, in particular a UVA screening agent, in which compositions the retinoid is not decomposed by the screening agent, is thus understood, and is an object of this invention.

### SUMMARY OF THE INVENTION

The inventors have discovered, with astonishment, that a given family of UVA screening agents can be introduced into compositions comprising a retinoid without resulting in the decomposition of the latter. These compositions can thus be stored for several months without experiencing a deterioration in their effectiveness.

### DETAILED DESCRIPTION OF THE INVENTION

The invention thus relates to a composition comprising, preferably in a physiologically acceptable medium, at least one retinoid and at least one benzotriazole silicone chosen from the benzotriazole silicones corresponding to one of the following formulae:

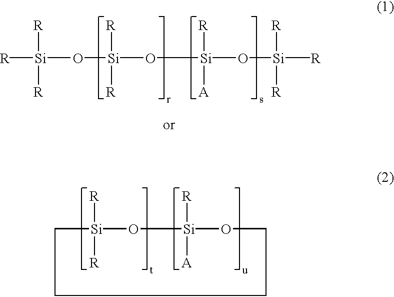

in which formulae (1) and (2)

R, which are identical or different, are chosen from C₁-C₁₀ alkyl, phenyl and 3,3,3-trifluoropropyl radicals, at least 80% by number of the R radicals being methyl,

r is an integer from 0 to 50 and s is an integer from 1 to 20,

u is an integer from 1 to 6 and t is an integer from 0 to 10, it being understood that t +u is equal to or greater than 3,

and the symbol A denotes a monovalent radical bonded directly to a silicon atom which corresponds to the following formula (3):

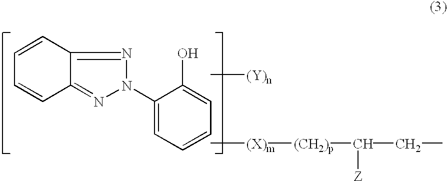

in which formula (3):

Y, which are identical or different, are chosen from C₁-C₈ alkyl radicals, halogens and C₁-C₄ alkoxy radicals, it being understood that, in the last case, two adjacent Y radicals of the same aromatic nucleus can together form an alkylidenedioxy group in which the alkylidene group comprises from 1 to 2 carbon atoms,

X represents O or NH,

Z represents hydrogen or a C₁-C₄ alkyl radical,

n is an integer from 0 to 3,

m is 0 or 1,

p represents an integer from 1 to 10.

As emerges from the formula (3) given above, the coupling of the —(X)m—(CH₂)ₚ— CH(Z)—CH₂— link to the benzotriazole unit, which thus provides the connection of the said benzotriazole unit to the silicon atom of the silicone chain, can, according to the present invention, take place at all the available positions offered by the two aromatic nuclei of the benzotriazole:

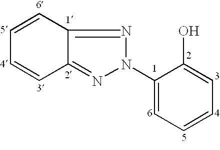

This coupling preferably takes place at the 3, 4, 5 (aromatic nucleus carrying the hydroxyl functional group) or 4′(benzene nucleus adjacent the triazole ring) position and more preferentially still at the 3, 4 or 5 position. In a preferred embodiment of the invention, the coupling takes place at the 3 position.

Likewise, the coupling of the Y substituent unit can take place at all the other available positions in the benzotriazole. However, preferably, this coupling takes place at the 3, 4, 4′, 5 and/or 6 position. In a preferred embodiment of the invention, the coupling takes place at the 5 position.

In the above formulae (1) and (2), the alkyl radicals can be linear or branched and can be chosen in particular from methyl, ethyl, n-propyl, isopropyl, n-butyl, isobutyl, tert-butyl, n-amyl, isoamyl, neopentyl, n-hexyl, n-heptyl, n-octyl, 2-ethylhexyl and tert-octyl radicals. The preferred R alkyl radicals according to the invention are the methyl, ethyl, propyl, n-butyl, n-octyl and 2-ethylhexyl radicals. More preferentially still, the R radicals are all methyl radicals.

It is preferable, among the compounds of above formulae (1) and (2), to make use of those corresponding to the formula (1), that is to say diorganosiloxanes comprising a short linear chain.

Preference is more particularly given, among linear diorganosiloxanes coming within the scope of the present invention, to random derivatives or well-defined block derivatives exhibiting at least one, and more preferentially still the combination, of the following characteristics:

R is alkyl and more preferentially still is methyl,

r is from 0 to 5;

n is non-zero and preferably equal to 1 and Y is then chosen from methyl, tert-butyl or C₁-C₄ alkoxy,

Z is hydrogen or methyl,

m=0 or [m=1 and X=O],

p is equal to 1

A family of compounds particularly suitable for the invention is that defined by the following general formula:

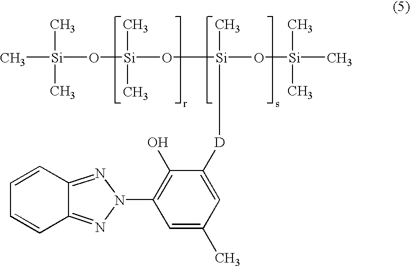

with

0≦r≦15, preferably 0≦r≦10,

1≦s≦5, preferably 1≦s≦3

and where D represents the divalent radical:

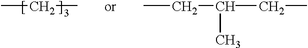

In a particularly preferred embodiment of the invention, the benzotriazole silicone corresponds to the following general formula (6):

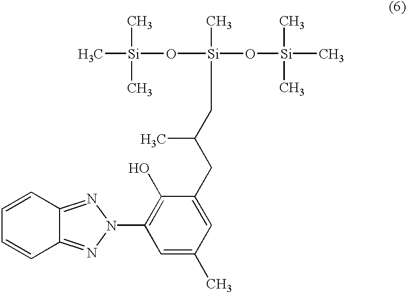

These benzotriazole silicones, and their method of preparation, are disclosed in particular in Application FR-A-2 642 968 incorporated herein by reference. One of ordinary skill in the art can obtain or make these compounds given the disclosure herein. They exhibit good screening properties in a wide range of wavelengths from 280 nm to 360 nm and good cosmetic properties because of their ready solubility in fatty substances.

The retinoid according to the invention can be retinol, retinal or a retinyl ester, such as an ester of retinol and of a C₂-C₂₀ acid, such as retinyl propionate, acetate or palmitate.

According to a preferred embodiment of the invention, the retinoid is retinol. This term is understood to mean all the isomers of retinol, namely all-trans-retinol, 13-cis-retinol, 11-cis-retinol, 9-cis-retinol and 3,4-didehydroretinol. Preferably, all-trans-retinol is used.

The composition according to the invention is preferably intended for a cosmetic or dermatological use, advantageously cosmetic use. If intended for a topical application to the skin it thus generally comprises a physiologically acceptable medium, that is to say a medium compatible with the skin.

The composition according to the invention generally comprises an amount of retinoid which is effective in producing the desired effect, for example of between 0.01 and 0.2% by weight and preferably of between 0.01 and 0.15 by weight, with respect to the total weight of the composition. It additionally includes an amount of benzotriazole silicone which is sufficient to confer the desired Sun Protection Factor on it, for example from 0.5 to 15% by weight and preferably from 0.7 to 5% by weight of benzotriazole silicone, with respect to the total weight of the composition.

For better protection with respect to UV radiation, the composition according to the invention can, in addition to the abovementioned at least one benzotriazole silicone, include at least one other compound capable of screening out UVA radiation and/or at least one other compound capable of screening out UVB radiation and/or at least one optionally coated inorganic pigment.

Mention may in particular be made, as other compound (non-benzotriazole silicone) capable of screening out UVA radiation, of: (1) benzophenone derivatives, for example:

2,4-dihydroxybenzophenone (benzophenone-1);

2,2′, 4,4′-tetrahydroxybenzophenone (benzophenone-2);

2-hydroxy-4-methoxybenzophenone (benzophenone-3), available from BASF under the trade name UVINUL M40;

2-hydroxy-4-methoxybenzophenone-5-sulphonic acid (benzophenone-4) and its sulphonate form (benzophenone-5), available from BASF under the trade name UVINUL MS40;

2,2′-dihydroxy-4,4′-dimethoxybenzophenone (benzophenone-6);

5-chloro-2-hydroxybenzophenone (benzophenone-7);

2,2′-dihydroxy-4-methoxybenzophenone (benzophenone-8);

the disodium salt of 2,2′-dihydroxy-4,4′-dimethoxybenzophenone-5,5′-disulphonic diacid (benzophenone-9);

2-hydroxy-4-methoxy-4′-methylbenzophenone (benzophenone-10);

benzophenone-11;

2-hydroxy-4-(octyloxy)benzophenone (benzophenone-12), benzophenone-3 and benzophenone-5 being preferred;

(2) triazine derivatives and in particular 2,4-bis[4-(2-ethylhexyloxy)-2-hydroxyphenyl]-6-(4-methoxyphenyl)-1,3,5-triazine, available from Ciba-Geigy under the trade name Tinosorb S, and 2,2′-methylenebis[6-(2H-benzotriazol-2-yl)-4-(1,1,3,3-tetramethylbutyl)phenol], available from Ciba-Geigy under the trade name Tinosorb M;

(3) screening agents which are active in the UV-A region of following formula (I):

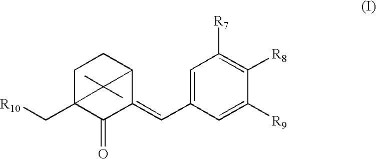

in which:

R₇ and R₉, which are identical or different, denote a hydrogen, a halogen, an OH radical, a saturated or unsaturated and linear or branched Cl-C₁-C₁₀ alkyl radical, a saturated or unsaturated and linear or branched C₁-C₁₀ alkoxy radical or an HSO₃ group;

R₁₀ denotes hydrogen or HSO₃;

R₈ denotes hydroxyl; or an OR₁₁ group where R₁₁ denotes a saturated or unsaturated and linear or branched C₁-C₁₀ alkyl radical; or else a group with the following structure:

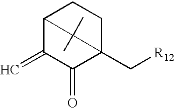

in which R₁₂ denotes hydrogen or HSO₃ and attachment is via the vinylene CH(═CH); or else a group with the following formula:

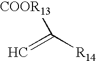

where attachment is via the vinylene CH(═CH), or else a group with the following formula:

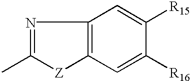

in which groups:

Z denotes an oxygen atom or an —NH— radical;

R₁₃, R₁₄, R₁₅ and R₁₆, which are identical or different, denote a hydrogen, a halogen, an OH radical, a saturated or unsaturated and linear or branched C₁-C₁₀ alkyl radical, a saturated or unsaturated and linear or branched C₁-C₁₀ alkoxy radical or an HSO₃ group, in particular benzene-1,4-di(3-methylidenecamphor-10-sulphonic acid) or one of its alkali metal, alkaline earth metal or ammonium salts or one of its salts with an organic base, which corresponds to the following formula:

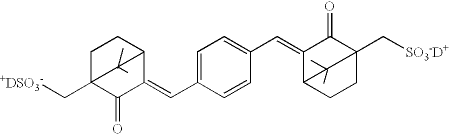

in which D denotes a hydrogen atom, an alkali metal, an NH(R₂₅)₃⁺ radical, in which the R₂₅ radicals, which can be identical or different, denote a hydrogen atom or a C₁-C₄ alkyl or hydroxyalkyl radical, or an Mⁿ⁺/n group, Mⁿ⁺ denoting a polyvalent metal cation in which n is equal to 2 or 3 or 4, Mⁿ⁺ preferably denoting a metal cation chosen from Ca²⁺, Zn²⁺, Mg²⁺, Ba²⁺, Al³⁺ and Zr⁴⁺, (4) their mixtures.

Mention may in particular be made, as other compounds capable of screening out UVB radiation, of: (1) salicylic acid derivatives, in particular homomethyl salicylate and octyl salicylate; (2) cinnamic acid derivatives, in particular 2-ethyl-hexyl p-methoxycinnamate, available from Givaudan under the trade name Parsol MCX;

(3) liquid β,β′-diphenylacrylate derivatives, in particular 2-ethylhexyl α-cyano-β,β′-diphenylacrylate, or octocrylene, available from BASF under the trade name Uvinul N539;

(4) p-aminobenzoic acid derivatives;

(5) 4-methylbenzylidenecamphor, available from Merck under the trade name Eusolex

(6) 2-phenylbenzimidazole-5-sulphonic acid, sold under the trade name “Eusolex 232” by Merck,

(7) 1,3,5-triazine derivatives, in particular: -2,4,6-tis[p-(2′-ethylhexyl-1′-oxy-carbonyl)anilino]-1,3,5-triazine, available from BASF under the trade name Uvinul T150, and

the compound corresponding to the following formula:

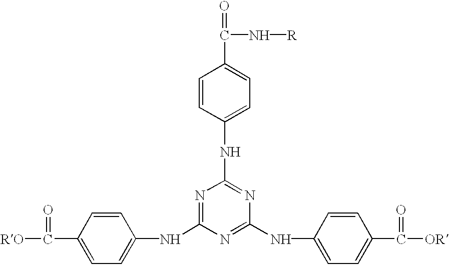

in which R′ denotes a 2-ethylhexyl radical and R denotes a tert-butyl radical, available from Sigma 3V under the trade name Uvasorb HEB;

(8) their mixtures.

Some optionally coated inorganic pigments which can be used in the composition according to the invention include coated or noncoated metal oxide nanopigments (mean size of the primary particles: generally between 5 nm and 100 nm, preferably between 10 nm and 50 nm), such as, for example, titanium oxide (amorphous or crystallized in the rutile and/or anatase form), iron oxide, zinc oxide, zirconium oxide or cerium oxide nanopigments, which are all UV photoprotective agents well known per se. Conventional coating agents are, furthermore, alumina and/or aluminum stearate. Such coated or noncoated metal oxide nanopigments are disclosed in particular in Patent Applications EP-A-0 518 772 and EP-A-0 518 773, incorporated herein by reference.

The composition according to the invention can be provided in any pharmaceutical dosage form normally used in cosmetic and dermatological fields and it can in particular be in the form of an optionally gelled aqueous solution, of a dispersion of the lotion type, optionally with two phases, of an emulsion obtained by dispersion of a fatty phase in an aqueous phase (O/W) or vice versa (W/O), of a triple emulsion (W/O/W or O/W/O) or of a vesicular dispersion of ionic and/or nonionic type. These composition forms are prepared according to conventional methods. Preferably, the composition according to the invention is in the form of an oil-in-water emulsion.

Compositions of the invention can be more or less fluid and have the appearance of a white or colored cream, of an ointment, of a milk, of a lotion, of a serum, of a paste or of a foam. It can also be applied to the skin in the aerosol form. It can also be provided in the solid form, in particular in the form of a stick for the lips. It can be used as care product and/or as make-up product for the skin.

The compositions of the invention can also comprise adjuvants conventional in the cosmetics field, such as hydrophilic or lipophilic gelling agents, hydrophilic or lipophilic active principles, preservatives, antioxidants, solvents, fragrances, fillers, pigments, odor absorbers and coloring materials. The amounts of these various adjuvants are those conventionally used in the field under consideration, for example from 0.01 to 20% of the total weight of the composition. These adjuvants, depending on their nature, can be introduced into the fatty phase, into the aqueous phase or into the lipid vesicles. In any event, these adjuvants, and their proportions, will be chosen so as not to harm the desired properties of the combination according to the invention.

When the composition of the invention is an emulsion, the proportion of the fatty phase can range from 5 to 80% by weight and preferably from 5 to 50% by weight with respect to the total weight of the composition. The oils, the emulsifiers and the coemulsifiers used in the composition in the emulsion form are chosen from those conventionally used in the field under consideration. The emulsifier and the coemulsifier are present in the composition in a proportion ranging from 0.3 to 30% by weight and preferably from 0.5 to 20% by weight, with respect to the total weight of the composition.

Mention may be made, as oils which can be used in the invention, of mineral oils (liquid petrolatum), oils of vegetable origin (avocado oil, soybean oil), oils of animal origin (lanolin), synthetic oils (perhydrosqualene), silicone oils (cyclomethicone) and fluorinated oils (perfluoropolyethers). Use may also be made, as fatty substances, of fatty alcohols (cetyl alcohol), fatty acids or waxes (carnauba wax, ozokerite).

Mention may be made, as emulsifiers and coemulsifiers which can be used in the invention, of, for example, polyethylene glycol fatty acid esters, such as PEG-100 stearate, and glycerol fatty acid esters, such as glyceryl stearate.

Mention may in particular be made, as hydrophilic gelling agents, of carboxyvinyl polymers (carbomer), acrylic copolymers, such as acrylate/alkylacrylate copolymers, polyacrylamides, polysaccharides, natural gums and clays and mention may be made, as lipophilic gelling agents, of modified clays, such as bentones, metal salts of fatty acids, hydrophobic silica and polyethylenes.

The present invention also relates to the preparation of the disclosed compositions by adding together, mixing, adding one to the other, etc., hereinafter termed “contacting” a retinoid and benzotriazole silicone, and to cosmetic uses of the composition described above, in particular for preventing or treating signs of intrinsic or photoinduced cutaneous ageing. Generally speaking, the compositions of the invention are applied to the skin, lips, scalp, etc. in a conventional manner.

The invention also relates to the use of the composition described above for manufacturing a preparation intended to prevent or treat cutaneous damage due to UV radiation.

The invention will be better understood and its advantages will more clearly emerge in the light of the following examples, which are given by way of illustration and without limitation.

### EXAMPLES

### Example 1

### Cosmetic Composition

| Phase A | |
|---|---|
| Glyceryl stearate and PEG-100 stearate | 2.1% |
| Polysorbate 60 | 0.9% |
| Cetyl alcohol | 2.6% |
| Hydrogenated polyisobutene | 12% |
| Hexyldecanol | 8% |
| BHT | 0.1% |
| Preservative | 0.15% |
| Benzotriazole silicone of formula (6) | 3% |
| Phase B | |
| Water | q.s. for 100% |
| Glycerol | 3% |
| Preservative | 0.55% |
| Pentasodium salt of ethylenediamine- | 0.07% |
| tetramethylenephosphonic acid | |
| Phase C | |
| Xanthan gum | 0.1% |
| Carbomer | 0.4% |
| Phase D | |
| Water | 5% |
| Triethanolamine | 0.38% |
| Phase E | |
| Retinol | 0.1% |

The above composition can be prepared in the following way.

Phases A and B are heated separately with stirring at 75° C. until completely dissolved. Phase D is prepared separately with stirring at ambient temperature. Phase A is subsequently decanted into phase B with stirring at 75° C. over 5-10 min and then the combined mixture is cooled to 50° C. The constituents of phase C are subsequently introduced into the mixture of phases A and B with stirring at 50° C. and, after complete homogenization, phase D is successively added with stirring. The mixture is cooled to ambient temperature. After rendering inert with nitrogen, phase E is introduced into the vessel with stirring.

This composition can be used daily as a day cream for preventing and combating wrinkles and for toning up the skin.

### Example 2

### Demonstration of the Stabilization of the Retinol

The stability of the retinol was evaluated in the three compositions A to C below:

Composition A: Composition of Example 1 without the benzotriazole silicone,

Composition B: Composition of Example 1,

Composition C: Composition of Example 1 in which the benzotriazole silicone is replaced by 3%- of 4-tertbutyl-4′-methoxydibenzoylmethane (sold under the name Parsol 1789 by Givaudan) in phase A.

The amounts by weight of retinol respectively present in compositions A to C above were determined, first, at ambient temperature immediately after preparation (To) of these compositions and, secondly, after storage at 45° C. for two months (T2m).

These measurements were made by High Pressure Liquid Chromatography using, as standard, a 16 μg/ml solution of retinol in THF prepared from a sample of Retinol 10S sold by BASF in the form of a 10% oily solution of retinol.

The results obtained are as follows:

| | To | To |
|---|---|---|
| Composition A | 0.108% | 0.102% |
| Composition B | 0.106% | 0.103% |
| Composition C | 0.105% | 0.061% |

Taking into account the accuracy of the method of quantitative determination used, it may be deduced therefrom that composition B, comprising the benzotriazole silicone as UVA screening agent, is as stable as composition A, not comprising a UV screening agent, and markedly more stable than composition C, comprising another UVA screening agent.

French patent application 0013938 filed Oct. 30, 2000 is incorporated herein by reference.

1. A composition comprising at least one retinoid and at least one benzotriazole silicone selected from the group consisting of benzotriazole silicones corresponding to one of the following formulae (1) and (2): 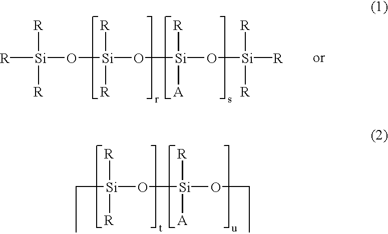
in which formulae (1) and (2):
R, which are identical or different, are chosen from C₁-C₁₀ alkyl, phenyl and 3,3,3-trifluoropropyl radicals, at least 80% by number of the R radicals being methyl,
r is an integer from 0 to 50 and s is an integer from 1 to 20,
u is an integer from 1 to 6 and t is an integer from 0 to 10, t+u is equal to or greater than 3,
and the symbol A denotes a monovalent radical bonded directly to a silicon atom which corresponds to the following formula (3): 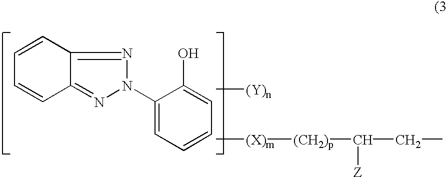
in which formula (3):
Y, which are identical or different, are chosen from C₁-C₈ alkyl radicals, halogens and C₁-C₄ alkoxy radicals, and where two adjacent Y radicals of the same aromatic nucleus can together form an alkylidenedioxy group in which the alkylidene group comprises from 1 to 2 carbon atoms,
X represents 0 or NH,
Z represents hydrogen or a C₁-C₄ alkyl radical,
n is an integer from 0 to 3,
m is 0 or 1,
p represents an integer from 1 to 10.

2. The composition according to claim 1, comprising a benzotriazole silicone compound of general formula (1) exhibiting at least one of the following characteristics:
R is alkyl;
r is from 0 to 15; s is from 1 to 5,
n is non-zero and Y is selected from the group consisting of methyl, tert-butyl or C₁-C₄ alkoxy,
Z is hydrogen or methyl,
m=0 or m=1 and Y is oxygen,
p is equal to 1.

3. The composition according to claim 2, comprising a benzotriazole silicone compound of general formula (5): 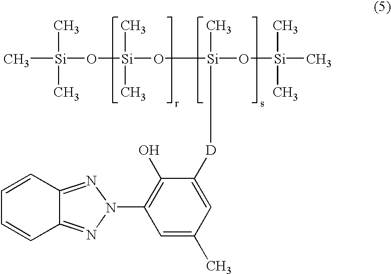
with
0≦r≦15,
1≦s≦5
and where D represents the divalent radical:

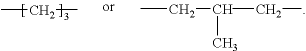

4. The composition according to claim 3, comprising a benzotriazole silicone compound of formula (6):

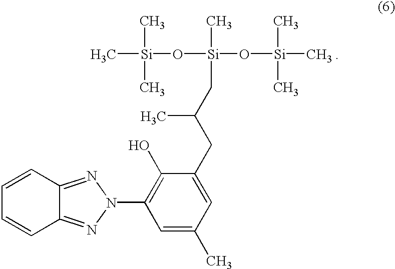

5. The composition according to claim 1, wherein said retinoid is selected from the group consisting of retinol, retinal and retinyl esters.

6. The composition according to claim 1, comprising a retinyl ester selected from the group consisting of esters of retinol with a C₂-C₂₀ acid.

7. The composition according to claim 1, comprising retinol.

8. The composition according to claim 1, comprising from 0.01 to 0.15% by weight of said retinoid with respect to the total weight of the composition.

9. The composition according to claim 1, comprising from 0.7 to 5% by weight of said benzotriazole silicone with respect to the total weight of the composition.

10. The composition according to claim 1, wherein said composition is in the form of an oil-in-water emulsion.

11. The composition according to claim 1, further comprising at least one non-benzatriazole silicone compound capable of screening out UVA radiation and/or at least one non-benzatriazole silicone other compound capable of screening out UVB radiation and/or at least one optionally coated inorganic pigment.

12. The composition according to claim 1, further comprising a compound capable of screening out UVA radiation selected from the group consisting of
(1) benzophenone derivatives,
(2) triazine derivatives,
(3) benzene-1,4-di(3-methylidene-10-camphorsulphonic acid) or one of its alkali metal, alkaline earth metal or ammonium salts or one of its salts with an organic base; and
(4) mixtures thereof.

13. The composition according to claim 1, further comprising a compound capable of screening out UVA radiation selected from the group consisting of
2-hydroxy-4-methoxybenzophenone (benzophenone-3), 2-hydroxy-4-methoxybenzophenone-5-sulphonic acid, 2,4-bis[4-(2-ethylhexyloxy)-2-hydroxyphenyl]-6-(4-methoxyphenyl)-1,3,5-triazine, 2,2′-methylene-bis[6-(2H-benzotriazol-2-yl)-4-(1,1,3,3-tetramethylbutyl)phenol], and benzene-1,4-di(3-methylidene-10-camphorsulphonic acid) or one of its alkali metal, alkaline earth metal or ammonium salts or one of its salts with an organic base.

14. The composition according to claim 1, further comprising a compound capable of screening out UVB radiation selected from the group consisting of
(1) salicylic acid derivatives,
(2) cinnamic acid derivatives,
(3) liquid βP,β′-diphenylacrylate derivatives,
(4) p-aminobenzoic acid derivatives;
(5) 4-methylbenzylidenecamphor;
(6) 2-phenylbenzimidazole-5-sulphonic acid;
(7) 1,3,5-triazine derivatives, and
(8) mixtures thereof.

15. The composition according to claim 1, further comprising a compound capable of screening out UVB radiation selected from the group consisting of
(1) homomethyl salicylate and octyl salicylate,
(2) 2-ethylhexyl p-methoxycinnamate,
(3) 2-ethylhexyl α-cyano-β,β′-diphenylacrylate, or octocrylene,
(4) 4-methylbenzylidenecamphor,
(5) 2-phenylbenzimidazole-5-sulphonic acid,
(6) 2,4,6-tris[p-(2′-ethylhexyl-1′-oxycarbonyl)anilino]-1,3,5-triazine and, the compound corresponding to the following formula: 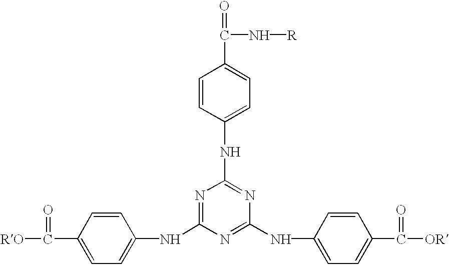
in which R′ denotes a 2-ethylhexyl radical and R denotes a tert-butyl radical.

16. The composition according to claim 1, further comprising an inorganic pigment selected from the group consisting of titanium oxide, iron oxide, zinc oxide, zirconium oxide or cerium oxide nanopigments optionally coated with alumina and/or with aluminum stearate.

17. A method of treating or preventing signs of intrinsic or photoinduced ageing comprising applying the composition of claim 1 to the skin, lips, or scalp.

18. A method for preparing the composition of claim 1, comprising contacting said retinoid and benzotriazole silicone.
